(19) United States
(12) Patent Application Publication
(10) Pub. No.: US 2025/0198895 A1
(43) Pub. Date: Jun. 19, 2025

(54) APPARATUS AND METHOD FOR MEASURING CONCRETE SLUMP AND SLUMP FLOW

(71) Applicant: ECODAX CO., LTD., Yunlin County (TW)

(72) Inventor: Ching-Cheng LAI, Yunlin County (TW)

(21) Appl. No.: 18/985,603

(22) Filed: Dec. 18, 2024

(30) Foreign Application Priority Data
Dec. 19, 2023 (TW) 112149509

(51) Int. Cl. G01N 11/02; G01N 11/00; G01N 33/38

(52) U.S. Cl. CPC G01N 11/02; G01N 33/383; G01N 2011/0026

(57) ABSTRACT

Provided is an apparatus and method for measuring slump and slump flow of concrete, including introducing concrete into a feed tank, opening a feed valve to allow the concrete to fall into a slump cone, and controlling, by a slump plate operation mechanism, the slump cone carried by the slump plate to move away from the feed valve. A slump cone operation mechanism controls the slump cone to move away from the slump plate along a direction perpendicular to the slump plate, leaving the concrete on the slump plate. A slump and slump flow measurement mechanism projects a slump/slump flow marker onto the concrete left on the slump plate to capture an image of the concrete and the slump/slump flow markers. The captured image is output to a processor to calculate the slump/slump flow of the concrete.

### CROSS-REFERENCE TO RELATED APPLICATIONS

This application is a U.S. non-provisional application filed under 35 U.S.C. § 111(a), which claims benefit to the priority of Taiwan Patent Application No. 112149509, filed on Dec. 19, 2023, the contents of which are hereby incorporated by reference in its entirety.

### TECHNICAL FIELD

The present disclosure relates to technologies for measuring slump and slump flow, and more particularly to an apparatus and method for measuring concrete slump and slump flow.

### BACKGROUND

Concrete is one of the primary engineering materials for constructing structures, and its quality significantly impacts the safety and usability of these structures. At present, concrete is mostly processed using a pre-mix approach, where it is first mixed at a pre-mix plant and then transported to construction sites by concrete trucks for direct pumping and placement. However, as a composite material, the final strength of concrete can be directly affected by various factors. In particular, the requirements for slump and slump flow are fundamental indicators of concrete workability. For example, insufficient or excessive slump/slump flow can impact the future strength of the structure. Moreover, the required slump/slump flow often varies depending on the specific environmental conditions.

Currently, the testing of concrete slump and slump flow remains at a manual stage. The process involves first manually filling the slump cone by compacting the concrete in layers. After filling, the slump cone is lifted upward to leave a pile of concrete. The slump cone is then placed beside the concrete pile, and testing personnel utilizes a ruler to directly measure the height difference between the slump cone and the concrete pile. This height difference is referred to as the “slump,” while the extent of the concrete's spread is referred to as the “slump flow.”

However, there are numerous issues associated with manually testing the slump and slump flow of concrete. For instance, it is difficult to ensure that the slump cone is lifted vertically during manual operation, which may lead to disruption of the shape of the concrete pile before testing the slump and slump flow. Consequently, directly measuring the height difference (i.e., slump) and the flow spread (i.e., slump flow) using a ruler often results in significant errors. Furthermore, current manual testing methods are not conducive to repeated measurements of concrete slump and slump flow, leading to substantially increased time and labor costs. Additionally, the likelihood of measurement errors tends to rise with the increasing number of tests.

Hence, there is an urgent need in the relevant technical field for more convenient, efficient, and accurate apparatus and methods for measuring concrete slump and slump flow.

### SUMMARY

To address the aforementioned issues and other problems, the present disclosure provides an apparatus and method for measuring concrete slump and slump flow.

In at least one embodiment, the present disclosure provides an apparatus for measuring concrete slump and slump flow, comprising: a frame; a feed valve disposed within the frame; a feed tank disposed above the feed valve and configured to hold concrete to be measured; a slump cone disposed below the feed valve, the slump cone having an upper opening aligning with the feed valve, allowing the concrete to fall from the feed tank and pass through an opened feed valve into the slump cone; a slump plate disposed below the slump cone, the slump plate configured to seal a lower opening of the slump cone, thereby retaining the concrete within the slump cone; a slump cone operation mechanism disposed on the frame and connected to the slump cone, the slump cone operation mechanism configured to move the slump cone away from the slump plate along a direction perpendicular to the plane of the slump plate, allowing the concrete to leave the slump cone and remain on the slump plate; a slump and slump flow measurement mechanism configured to project a marker onto the concrete remaining on the slump plate and to capture images of the concrete and the marker so as to output the images; and a slump plate operation mechanism connected to the slump plate and configured to tilt the slump plate, allowing the concrete to leave the slump plate.

In at least one embodiment of the present disclosure, the slump and slump flow measurement mechanism comprises a slump marker projector configured to project a slump marker, a slump flow marker projector configured to project slump flow markers, a slump image capturer, and a slump flow image capturer, wherein the slump flow markers are two straight lines intersecting each other perpendicularly and intersecting side edges of the concrete remaining on the slump plate, respectively, and the slump marker is a straight line intersecting an upper-end edge of the concrete remaining on the slump plate.

In at least one embodiment, the apparatus for measuring concrete slump and slump flow as disclosed in the present disclosure further comprises a processor configured to receive images captured by the slump and slump flow measurement mechanism and to calculate the slump and/or slump flow of the concrete, respectively, based on the images with the concrete and the slump marker and the slump flow markers thereon.

In at least one embodiment, the slump cone operation mechanism disclosed in the present disclosure comprises a vertical actuator connected to the frame and having an actuating rod, and a horizontal actuator connected to the vertical actuator, wherein the horizontal actuator includes a rod member, and the slump cone is disposed with a support member. The rod member of the horizontal actuator is configured to contact the support member to support the slump cone, and the vertical actuator adjusts a length of the actuating rod to move the slump cone toward or away from the feed valve.

In at least one embodiment, the slump plate operation mechanism disclosed in the present disclosure comprises a central actuator connected to a position near the center of the slump plate and an edge actuator connected to an edge of the slump plate, wherein the slump plate operation mechanism moves the slump cone carried on the slump plate toward or away from the feed valve along a vertical direction by the central actuator and the edge actuator, such that the upper opening of the slump cone approaches or moves away from the feed valve.

In at least one embodiment, the apparatus for measuring concrete slump and slump flow as disclosed in the present disclosure further comprises a cleaning water pipe and a drying air pipe disposed on at least one side of the slump plate, and a discharge chute disposed adjacent to a corresponding side of the slump plate.

In at least one embodiment, the apparatus for measuring concrete slump and slump flow as disclosed in the present disclosure further comprises a mixing bucket disposed above the feed tank and a load cell disposed on the mixing bucket, wherein the volume of the feed tank is substantially equal to the volume of the slump cone.

In at least one embodiment, the apparatus for measuring concrete slump and slump flow as disclosed in the present disclosure further comprises a vibration assembly disposed on the slump cone, allowing the concrete to enter the slump cone from the feed tank and to be compacted by vibrations of the vibration assembly.

The present disclosure further provides a method for measuring concrete slump and slump flow by using the apparatus for measuring concrete slump and slump flow as disclosed above, comprising: introducing the concrete into the feed tank; controlling the feed valve to open, allowing the concrete to fall from the feed tank, pass through the opened feed valve, and enter the slump cone, wherein the slump plate seals the lower opening of the slump cone to retain the concrete within the slump cone; controlling, by the slump plate operation mechanism, the slump plate, allowing the slump cone carried on the slump plate to move away from the feed valve; controlling, by the slump cone operation mechanism, the slump cone, allowing the slump cone to move away from the slump plate along a direction perpendicular to the plane of the slump plate, and allowing the concrete to leave the slump cone and remain on the slump plate; controlling the slump and slump flow measurement mechanism to project a marker onto the concrete remaining on the slump plate; controlling the slump and slump flow measurement mechanism to capture images of the concrete and the markers, and outputting the captured images; and controlling, by the slump plate operation mechanism, the slump plate to tilt, allowing the concrete to leave the slump plate.

In at least one embodiment, the step of the present disclosure for controlling the slump and slump flow measurement mechanism to project markers onto the concrete remaining on the slump plate further comprises: employing the slump and slump flow measurement mechanism to project a slump marker and slump flow markers onto the concrete on the slump plate, wherein the slump flow markers are two straight lines intersecting each other perpendicularly and intersecting side edges of the concrete on the slump plate, respectively, and the slump marker is a straight line intersecting an upper-end edge of the concrete on the slump plate.

In at least one embodiment, the method for measuring concrete slump and slump flow as disclosed in the present disclosure further comprises: employing the slump and slump flow measurement mechanism to start capturing images when the slump cone moves away from the slump plate, and transmitting the captured images to the processor, allowing the processor to calculate the slump and/or slump flow of the concrete based on the images, and stopping capturing the images once the slump and/or the slump flow of the concrete no longer change.

In at least one embodiment, the step of the present disclosure for controlling the slump cone by the slump cone operation mechanism such that the slump cone moves away from the slump plate along a direction perpendicular to the plane of the slump plate comprises: subjecting the rod member of the horizontal actuator of the slump cone operation mechanism to contact the support member on the slump cone; and adjusting a length of the actuating rod of the vertical actuator of the slump cone operation mechanism, allowing the slump cone to move away from the slump plate.

In at least one embodiment, the method for measuring concrete slump and slump flow as disclosed in the present disclosure further comprises, after completing image capturing, controlling the cleaning water pipe to discharge water to rinse the slump plate, allowing the water and the concrete to flow into the discharge chute, and controlling the drying air pipe to discharge air to dry the slump plate.

In at least one embodiment, the method for measuring concrete slump and slump flow as disclosed in the present disclosure further comprises: after the concrete leaves the slump plate and the slump plate is rinsed and dried, controlling, by the slump plate operation mechanism, the slump plate to return from a tilted position to a horizontal position; controlling, by the slump plate operation mechanism, the slump plate to move toward the slump cone to seal the lower opening of the slump cone; and subjecting the rod member of the horizontal actuator of the slump cone operation mechanism to return to a position without contacting the support member on the slump cone.

In at least one embodiment, the method for measuring concrete slump and slump flow as disclosed in the present disclosure further comprises: mixing the concrete in the mixing bucket; and controlling the mixing bucket to tilt, allowing the concrete to fall into the feed tank.

In at least one embodiment, the time from when the concrete falls from the feed tank in a previous batch to when the concrete falls from the feed tank in the next batch can be approximately 65 seconds.

In at least one embodiment, the slump cone operation mechanism controls the slump cone to be lifted vertically upward while maintaining a horizontal position for a period of about 5 seconds.

In at least one embodiment, the method for measuring concrete slump and slump flow as disclosed in the present disclosure further comprises compacting the concrete entering the slump cone from the feed tank by vibrations of a vibration assembly disposed on the slump cone.

According to the above one or more embodiments, it can be understood that the present disclosure provides an apparatus and method for measuring concrete slump and slump flow with an automatically mechanical design. This design addresses issues such as potential inadequate or uneven filling of concrete into the slump cone during the manual operation of first loading concrete into the slump cone and then lifting it, as well as the tilting of the slump cone during manual lifting that can cause the concrete pile to become skewed. By resolving these issues, the measurement accuracy of slump and slump flow is improved. Additionally, with the use of the marker projectors and image capturers as disclosed in the present disclosure, the slump and slump flow markers can be directly projected onto the slump plate, enabling the processor to perform image recognition and calculations of the slump and slump flow. This further allows the processor to make real-time and appropriate adjustments to the concrete formulation, thereby reducing operational time and labor costs.

### BRIEF DESCRIPTION OF THE DRAWINGS

By reading the following detailed descriptions of specific embodiments and referring to the accompanying drawings, the present disclosure can be more fully understood.

FIGS. 1A, 1B, and 2 to 4 are schematic side views of a first embodiment of the apparatus for measuring concrete slump and slump flow as disclosed in the present disclosure.

FIGS. 5 and 6 are schematic top views of the first embodiment of the apparatus for measuring concrete slump and slump flow as disclosed in the present disclosure.

FIGS. 7 to 12 are schematic side views of a second embodiment of the apparatus for measuring concrete slump and slump flow as disclosed in the present disclosure.

FIG. 13 is a schematic flow diagram of the method for measuring concrete slump and slump flow as disclosed in the present disclosure.

### DETAILED DESCRIPTION OF EMBODIMENTS

This specification discloses some specific embodiments in detail to enable a person having ordinary skill in the art to utilize the disclosed embodiments based on the content of the present disclosure. Not all steps or features of the disclosed embodiments are discussed in exhaustive detail, as many steps or features will be apparent to a person having ordinary skill in the art based on the content of the present disclosure.

It should be noted that, as used in the present disclosure, the singular forms “a” and “the” include plural referents, unless explicitly and unequivocally limited to one referent. Unless otherwise expressly indicated by the context, the term “or” can be used interchangeably with “and/or.”

As used herein, the terms “comprising,” “having,” “including,” and “containing” should be interpreted as open-ended terms (i.e., meaning “including, but not limited to”), unless otherwise expressly stated in this disclosure.

The present disclosure will be described with reference to multiple embodiments; it should be understood that the following embodiments should not be construed as limiting the scope of the present disclosure.

### First Embodiment

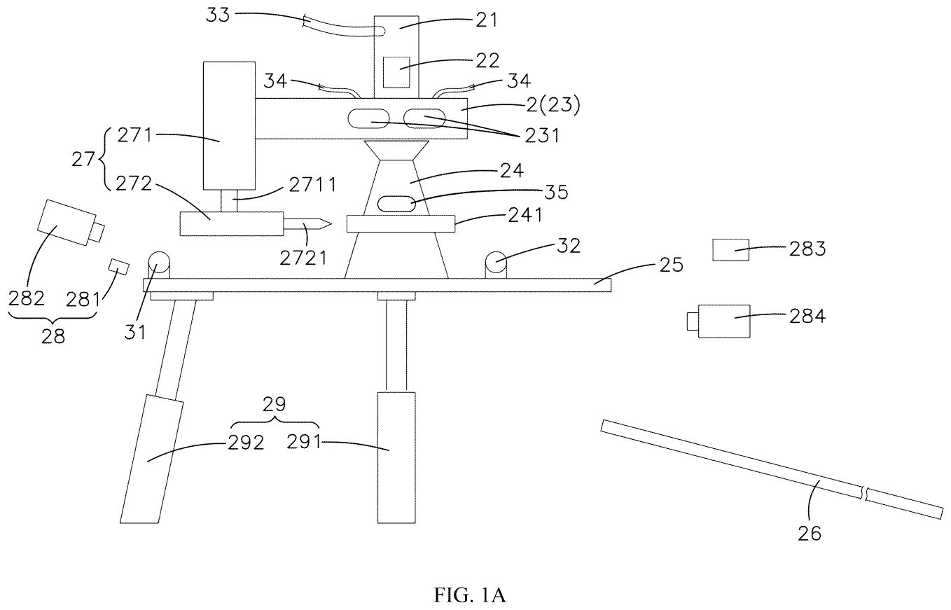

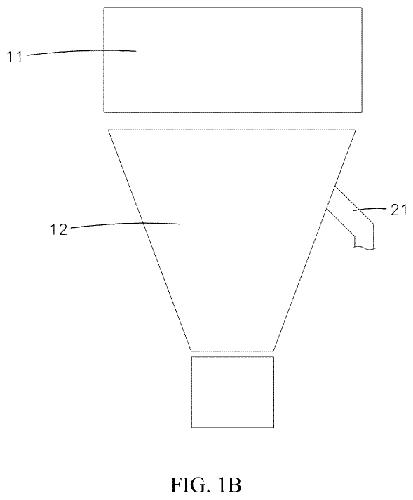

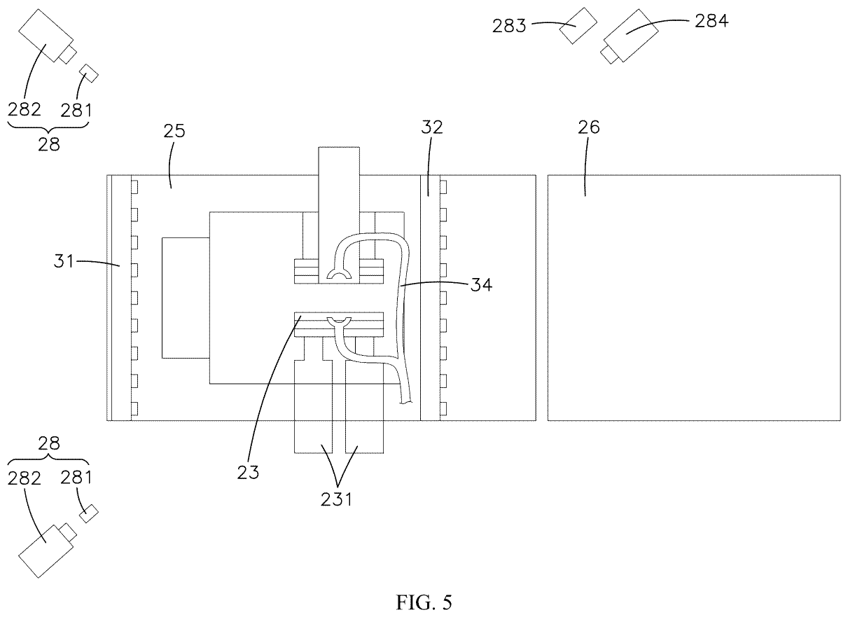

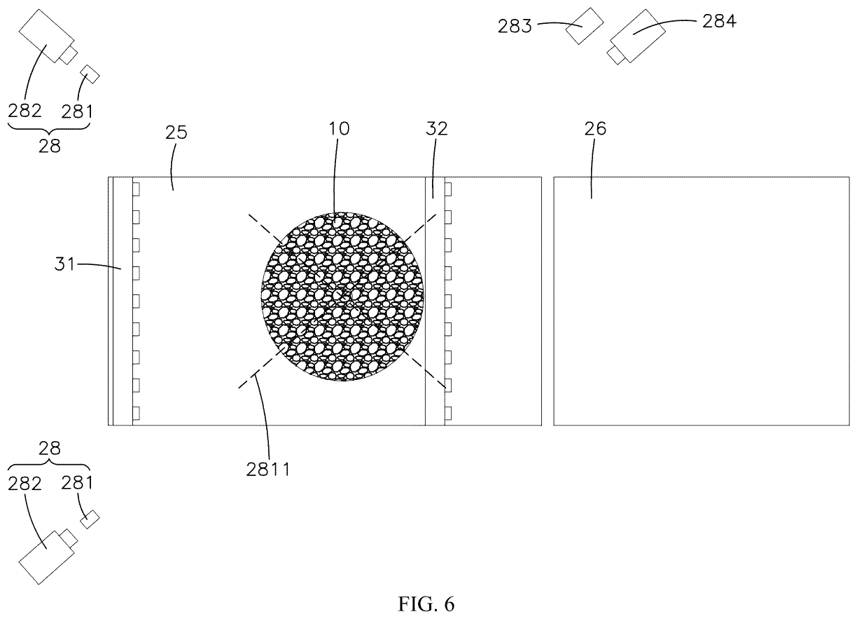

Please refer to FIGS. 1A, 1B, and 2 to 4, which illustrate schematic side views of one of the embodiments of the apparatus for measuring concrete slump and slump flow, and FIGS. 5 and 6, which illustrate schematic top views of the apparatus for measuring concrete slump and slump flow.

As shown in FIGS. 1A, 1B, and 2 to 4, the apparatus for measuring concrete slump and slump flow as disclosed in the present disclosure comprises a frame 2, a feed tank 21, a vibrator 22, a feed valve 23, a slump cone 24, a slump plate 25, a discharge chute 26, a slump cone operation mechanism 27, a slump and slump flow measurement mechanism 28, a slump plate operation mechanism 29, a cleaning water pipe 31, a drying air pipe 32, cleaning water tubes 33 and 34, and a vibration assembly 35.

In at least one embodiment of the present disclosure, the frame 2 is connected to the feed tank 21, so as to enable the input of concrete to be measured into the apparatus of this disclosure for measuring concrete slump and slump flow. In some embodiments of the present disclosure, the frame 2 may be provided with a controller for controlling the input of concrete, a monitor for observing the input conditions of concrete, and a sensor for detecting parameters such as temperature and humidity (not shown).

In at least one embodiment of the present disclosure, the feed tank 21 is disposed above the feed valve 23 within the frame 2 and configured to hold the concrete. For example, as shown in FIG. 1B, when the mixer 11 completes mixing the concrete, it discharges the pre-mixed concrete into both the storage hopper 12 and the feed tank 21. While the pre-mixed concrete in the storage hopper 12 is loaded into a pre-mixed concrete truck, the pre-mixed concrete also begins to be discharged from the feed tank 21 into the slump cone 24 via the feed valve 23. In some embodiments of the present disclosure, the mixer 11 provides pressure during discharging to ensure that the concrete is forced into the feed tank 21. In some embodiments of the present disclosure, the volume of the feed tank 21 is substantially equal to the volume of the slump cone 24, meaning that when the concrete enters the feed tank 21, the measured quantity matches the required testing amount. Accordingly, the structural volume and positional configuration of the feed tank 21 in the present disclosure enable automatic material retrieval.

In at least one embodiment of the present disclosure, a vibrator 22 is disposed on the feed tank 21, which generates vibrations as the concrete is discharged from the feed tank 21 into the slump cone 24. These vibrations ensure that the feed tank 21 vibrates, allowing the concrete to be evenly and properly loaded into the slump cone 24.

As shown in FIG. 5, in at least one embodiment of the present disclosure, the feed valve 23 disposed on the frame 2 can be opened or closed by using an actuator 231. In some embodiments of the present disclosure, the actuator 231 may, for example, be an oil pressure cylinder, a hydraulic cylinder, a pneumatic cylinder, or a motor, and the like. For instance, while concrete is being loaded into the feed tank 21, the feed valve 23 remains closed. When it is necessary to discharge the concrete from the feed tank 21 into the slump cone 24, the feed valve 23 opens, ensuring that the volume of concrete loaded into the slump cone 24 matches the required testing amount. In at least one embodiment of the present disclosure, the slump cone 24 has a conical shape. In some embodiments of the present disclosure, the slump cone 24 may alternatively have a cylindrical or columnar shape, and the present disclosure is not limited thereto.

In at least one embodiment of the present disclosure, the slump cone 24 is disposed below the feed valve 23, with the upper opening of the slump cone 24 being aligned with the feed valve 23, allowing the slump cone 24 to receive the concrete discharged from the feed tank 21 when the feed valve 23 is opened. This configuration enables automatic feeding into the slump cone 24. In some embodiments, the slump cone 24 of the present disclosure is disposed with a vibration assembly 35, which compacts the concrete within the slump cone 24 through vibrations generated by the vibration assembly 35.

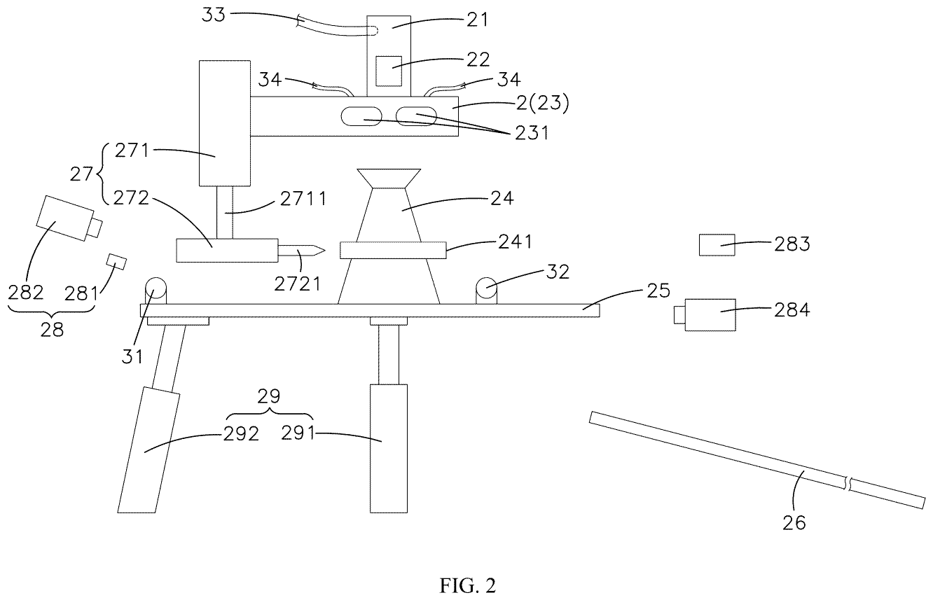

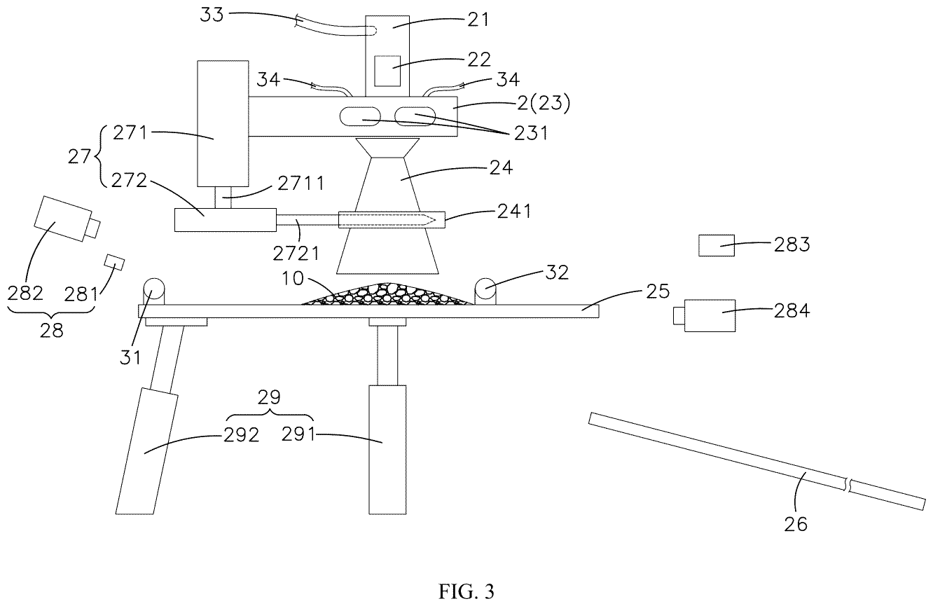

As shown in FIGS. 1A and 2, in at least one embodiment of the present disclosure, the slump plate 25 is disposed below the slump cone 24 and configured to support the slump cone 24 and seal its lower opening. This configuration ensures that the concrete discharged from the feed tank 21 into the slump cone 24 remains within the slump cone 24. In some embodiments of the present disclosure, when the slump cone 24 is lifted away from the slump plate 25, the concrete is left on the slump plate 25, as shown in FIG. 3.

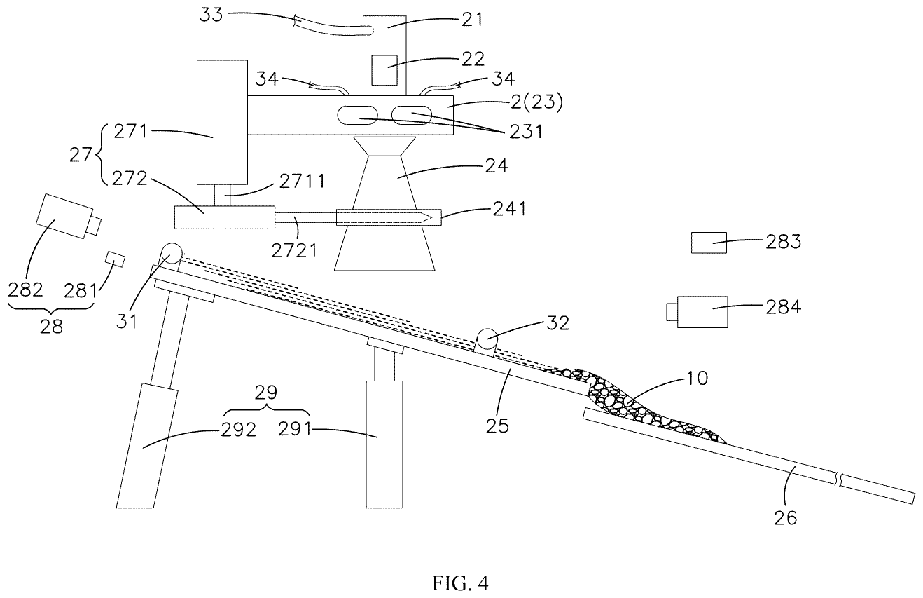

Moreover, as shown in FIG. 4, in at least one embodiment of the present disclosure, the discharge chute 26 is disposed on one side of the slump plate 25 and configured to receive the concrete discharged from the slump plate 25 when it is tilted.

Furthermore, as shown in FIGS. 1A, 1B, and 2 to 4, in at least one embodiment of the present disclosure, the slump cone operation mechanism 27 is connected to the frame 2 and comprises a vertical actuator 271 disposed on the frame 2 and a horizontal actuator 272 connected to the vertical actuator 271. In some embodiments of the present disclosure, the vertical actuator 271 and the horizontal actuator 272 may, for example, be oil pressure cylinders, hydraulic cylinders, pneumatic cylinders, or motors, and the like. For instance, during the loading of concrete into the slump cone 24, the rod member 2721 of the horizontal actuator 272 does not contact the support member 241 on the slump cone 24. However, after the concrete is loaded into the slump cone 24 and filling is complete, the rod member 2721 of the horizontal actuator 272 inserts horizontally into the support member 241 on the slump cone 24. The vertical actuator 271 then shortens its actuating rod 2711 to lift the slump cone 24 vertically, as shown in FIG. 3. In some embodiments of the present disclosure, the support member 241 contains two tubular components connected to both sides of the slump cone 24. The slump cone operation mechanism 27 is similar to a detachable fork frame or forklift. By incorporating the forklift-like design of the slump cone operation mechanism 27 and coupling with the structural design of the support member 241 on the slump cone 24, the slump cone 24 can be lifted vertically upward while maintaining a horizontal position, with the lifting process taking approximately 5 seconds. This achieves the effect of automatically lifting the slump cone 24.

As shown in FIG. 3, in at least one embodiment of the present disclosure, the slump and slump flow measurement mechanism 28 is configured to project markers onto the concrete 10 on the slump plate 25, capture images of the concrete 10 and the markers, and then output the captured images to a computer or a processor. Then, the computer utilizes image recognition technology to calculate the slump and slump flow of the concrete, wherein the slump and slump flow measurement mechanism 28 begins capturing images as the slump cone 24 moves away from the slump plate 25. The captured images are transmitted to the processor, which calculates the slump and slump flow of the concrete 10 based on the images. The image capturing process stops once the slump and slump flow of the concrete 10 no longer change. This configuration achieves the effect of automatically capturing images and calculating the slump and slump flow.

For example, in some embodiments of the present disclosure, the slump and slump flow measurement mechanism 28 comprises a slump flow marker projector 281 configured to project slump flow markers, a slump flow image capturer 282, a slump marker projector 283 configured to project a slump marker, and a slump image capturer 284.

As shown in FIGS. 5 and 6, in some embodiments of the present disclosure, the slump flow marker projector 281 is disposed near the slump plate 25 to project slump flow markers 2811 onto the concrete 10. In some embodiments of the present disclosure, the slump flow markers 2811 are two straight lines (e.g., two straight-line red laser beams) intersecting each other and intersecting with the side edges of the concrete 10 on the slump plate 25, respectively. The two straight lines are projected by two slump flow marker projectors 281, respectively. The slump flow image capturer 282 is disposed near the slump flow marker projector 281 to capture images of the concrete 10 and the slump flow markers 2811. In some embodiments of the present disclosure, the slump flow markers 2811 are two straight laser beams intersecting perpendicularly with each other and also intersecting perpendicularly with the tangents to the side edges of the concrete 10, enabling the measurement of two mutually perpendicular diameters of the concrete 10. For example, the slump flow markers 2811 are used to enhance the side edges of the concrete 10. When the processor receives the images captured by the slump flow image capturer 282, it uses image recognition technology to identify the side edges of the concrete 10 enhanced by the two straight lines of the slump flow markers 2811. By measuring the two diameters of the concrete 10 and calculating their average, the slump flow of the concrete 10 is determined. Thus, through the slump flow marker projector and the slump flow image capturer as disclosed in the present disclosure, the effectiveness of automatically measuring the slump flow of concrete is achieved.

In other embodiments of the present disclosure, the slump marker projector 283 further projects a slump marker, which is a straight line (e.g., a straight-line red laser beam) intersecting with the upper-end edge of the concrete 10 on the slump plate 25 and is projected by a slump marker projector 283. In some embodiments of the present disclosure, the slump marker can be used to enhance the upper-end edge of the concrete 10 to facilitate image recognition by a computer or a processor. Furthermore, the computer or the processor can measure the height difference of the upper-end edge of the concrete 10 before and after the slump cone 24 is lifted, thereby calculating the slump of the concrete. Thus, through the slump marker projector and the slump marker image capturer as disclosed in the present disclosure, the effectiveness of automatically measuring the concrete slump is achieved.

As shown in FIGS. 1A, 1B, and 2 to 4, the slump plate operation mechanism 29 of the present disclosure comprises a central actuator 291 disposed near the center of the slump plate 25 and an edge actuator 292 disposed at one side edge of the slump plate 25. In some embodiments of the present disclosure, the central actuator 291 and the edge actuator 292 jointly actuate the slump plate 25, enabling the slump plate 25 to ascend horizontally (as shown in FIG. 1A) or descend horizontally (as shown in FIG. 2). At the same time, the slump cone 24 carried by the slump plate 25 correspondingly ascends horizontally (as shown in FIG. 1A) or descends horizontally (as shown in FIG. 2). In some embodiments of the present disclosure, as shown in FIG. 4, when the extended length of the actuating rod of the edge actuator 292 exceeds that of the central actuator 291, the slump plate 25 tilts, allowing the concrete 10 on the slump plate 25 to slide into the discharge chute 26.

Similarly, as shown in FIG. 4, in at least one embodiment of the present disclosure, a cleaning water pipe 31 is additionally disposed on one side of the slump plate 25 opposite to the discharge chute 26 and configured to rinse the slump plate 25 and facilitate the sliding of the concrete 10 from the slump plate 25 into the discharge chute 26.

Furthermore, as shown in FIG. 5, in some embodiments of the present disclosure, a drying air pipe 32 may additionally be disposed on the side of the slump plate 25 opposite to the cleaning water pipe 31. In some embodiments of the present disclosure, the drying air pipe 32 may also be disposed on other sides of the slump plate 25, such as on both sides of the slump plate 25 opposite to the cleaning water pipe 31, to blow air for drying the slump plate 25. Thus, through the design of the slump plate operation mechanism 29, the cleaning water pipe 31, the drying air pipe 32, and the discharge chute 26 as disclosed in the present disclosure, the effectiveness of automatic cleaning of the slump plate 25 can be achieved. Additionally, in at least one embodiment of the present disclosure, the feed tank 21 and the slump cone 24 may be disposed with cleaning water pipes 33 and 34, respectively, so as to achieve the effectiveness of automatically cleaning the feed tank 21 and the slump cone 24, wherein the cleaning water pipe 33 is inserted into the feed tank 21 through a drilled hole from the outside, and the cleaning water pipe 34 is installed on the inner side of the iron frame supporting the support bearing of the pneumatic cylinder (i.e., the actuator of the feed valve 23). Furthermore, the cleaning water pipe 34 is smaller in size than the cleaning water pipe 33 and is provided in four units inside the iron frame (i.e., the frame 2) supporting the feed valve 23. However, the sizes and quantities of the cleaning water pipes 33 and 34 disclosed herein are not limited thereto.

As shown in FIGS. 1A, 1B, and 2 to 4, in at least one embodiment of the present disclosure, an image capturer (not shown) may be additionally disposed adjacent to the apparatus for measuring concrete slump and slump flow as disclosed in the present disclosure and configured to monitor the operation of measuring concrete slump and slump flow as disclosed in the present disclosure.

In at least one embodiment of the present disclosure, the apparatus for measuring concrete slump and slump flow completes a full process in approximately 65 seconds. For example, from the time the pre-mixed concrete is loaded from the storage hopper 12 into the pre-mixed concrete truck and simultaneously discharged from the feed tank 21 into the slump cone 24, to the subsequent steps including slump and slump flow measurement by the slump and slump flow measurement mechanism 28, cleaning, drying, and waiting for the next discharge into the next pre-mixed concrete truck while simultaneously discharging into the slump cone 24 for automatic sampling, the entire process requires no intervention from mixing personnel and takes only approximately 65 seconds.

### Second Embodiment

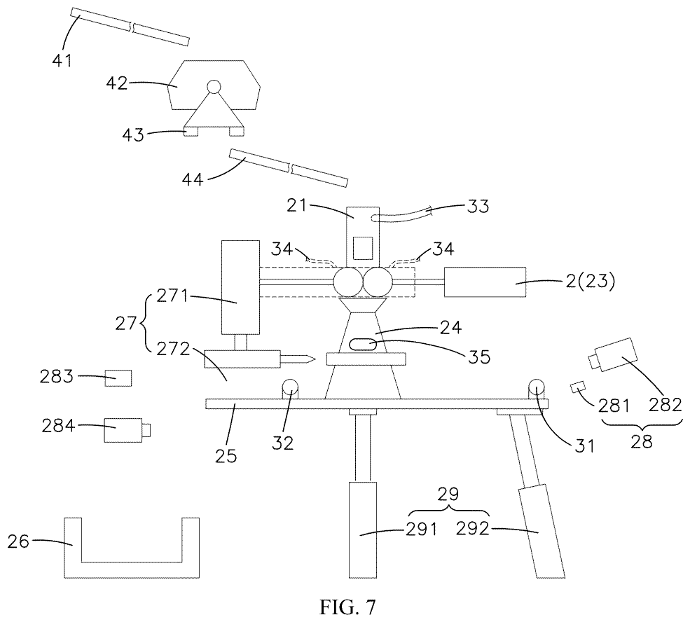

Please also refer to FIGS. 7 to 12, which illustrate schematic side views of the second embodiment of the apparatus for measuring concrete slump and slump flow.

In the first embodiment previously described, the apparatus for measuring concrete slump and slump flow as disclosed in the present disclosure is used while providing concrete to a pre-mixed concrete truck. In contrast to the first embodiment, the second embodiment simulates the conditions in which concrete is mixed within the mixing bucket of a pre-mixed concrete truck during transport of the concrete to a construction site. The simulation considers the time typically required for the transport journey, factoring in temperature effects, which may be, for example, 30 minutes, 45 minutes, 60 minutes, or 90 minutes. This simulation replicates the potential changes in the concrete within the mixing bucket of the pre-mixed concrete truck and records the workability of the concrete to enable real-time adjustments of the concrete slump and slump flow at the construction site.

As shown in FIG. 7, in at least one embodiment of the present disclosure, the concrete first enters the mixing bucket 42 through the feed track 41. In some embodiments of the present disclosure, a load cell 43 may additionally be disposed in the mixing bucket 42 to sense whether the input of concrete has reached the required sampling amount and to stop feeding once the required sampling amount is achieved.

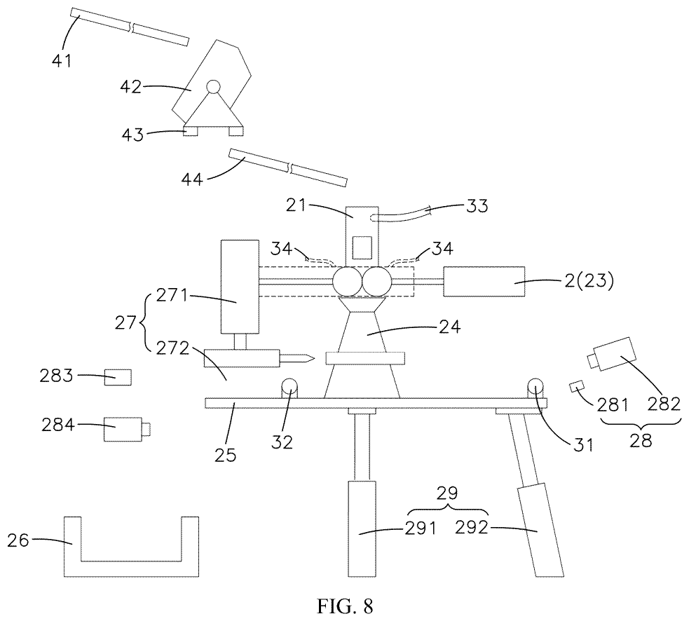

As shown in FIG. 8, in at least one embodiment of the present disclosure, the mixing bucket 42 simulates the mixing of concrete by a concrete mixer truck for a duration that may be, for example, 30 minutes, 45 minutes, 60 minutes, or 90 minutes, which can be appropriately adjusted based on actual requirements and is not specifically limited in the present disclosure. In some embodiments of the present disclosure, when the simulated mixing duration ends, the concrete is transferred to the feed tank 21 via the feed track 44.

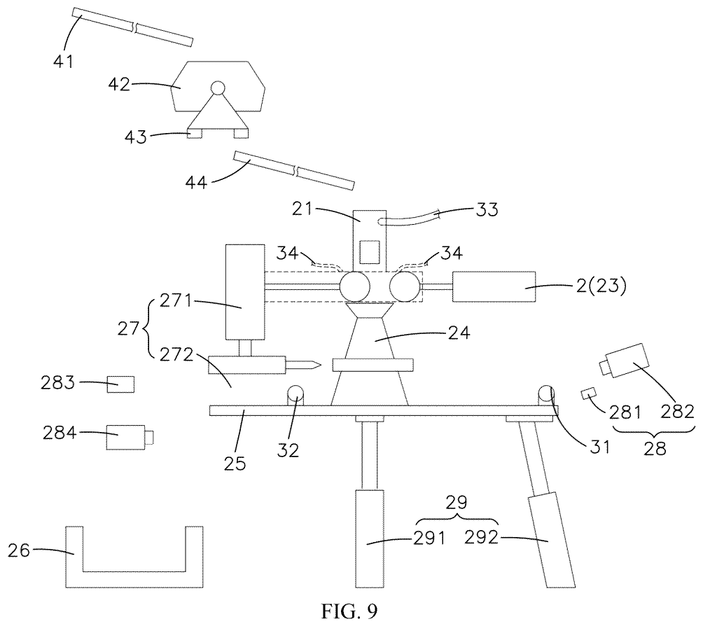

As shown in FIG. 9, in at least one embodiment of the present disclosure, the concrete falls into the slump cone 24 through the feed valve 23 after the feed valve 23 is opened.

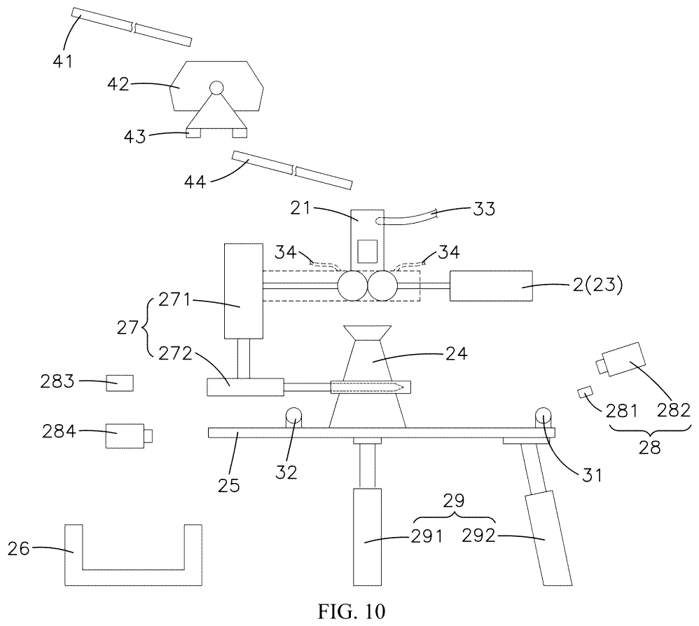

As shown in FIG. 10, in at least one embodiment of the present disclosure, the feed valve 23 closes after the concrete fills the slump cone 24. Subsequently, the slump plate operation mechanism 29 controls the slump plate 25 carrying the slump cone 24 to descend.

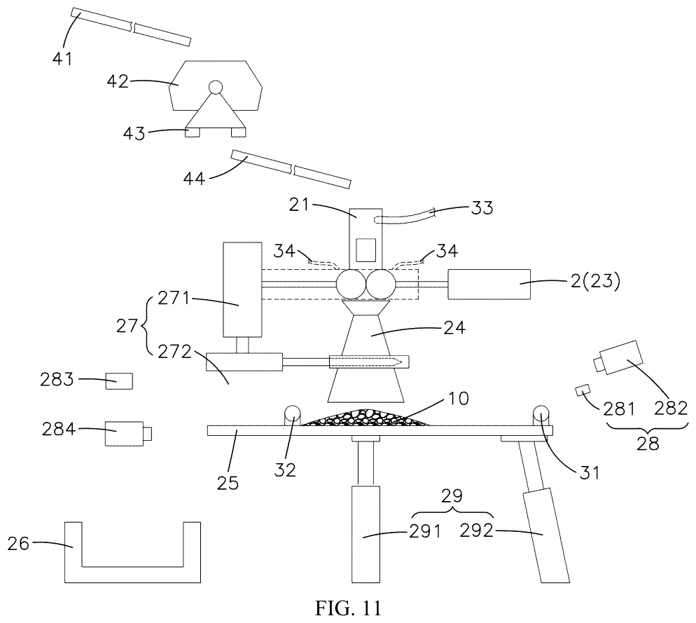

As shown in FIG. 11, in at least one embodiment of the present disclosure, the slump cone operation mechanism 27 controls the slump cone 24 to move upward, wherein the slump cone 24 is lifted vertically upward while maintaining a horizontal position, with the lifting process taking approximately 5 seconds. In addition, during this time, the slump and slump flow measurement mechanism 28 begins capturing images, which are processed by the processor for image recognition to measure the slump and slump flow. The captured image information and the measured slump and slump flow data are then transmitted to the computer at the mixing station, allowing mixing personnel to obtain real-time information of the concrete.

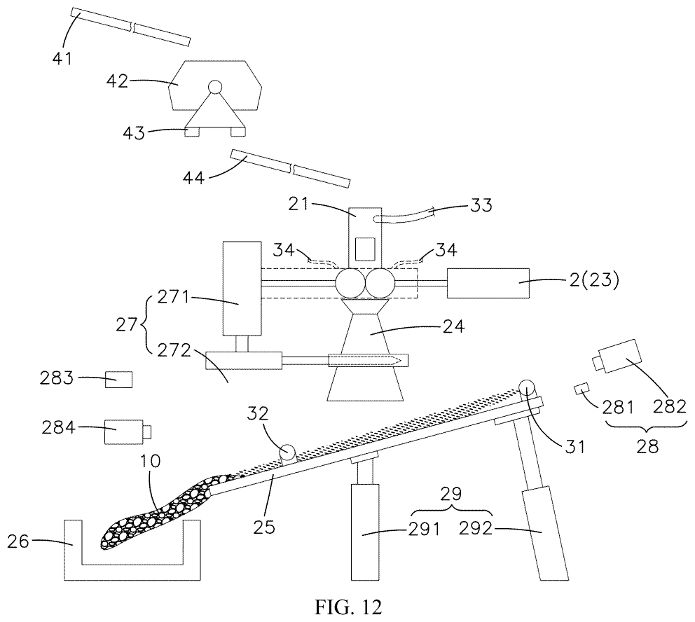

As shown in FIG. 12, in at least one embodiment of the present disclosure, after completing the measurement of the slump and slump flow, the slump plate operation mechanism 29 controls the slump plate 25 to tilt, allowing the concrete to slide into the discharge chute 26. In some embodiments of the present disclosure, the cleaning water pipe 31 then sprays a water jet to clean the slump plate 25. In some embodiments of the present disclosure, the drying air pipe 32 subsequently discharges gas to dry the slump plate 25. In some embodiments of the present disclosure, the gas discharged from the drying air pipe 32 may be pressurized air, heated air, or a combination thereof, so as to effectively dry the slump plate 25. Similarly, as shown in FIG. 12, in at least one embodiment of the present disclosure, cleaning water pipes 33 and 34 are disposed for the feed tank 21 and the slump cone 24, respectively, so as to achieve the cleaning of the feed tank 21 and the slump cone 24, wherein cleaning water pipe 34 is smaller in size compared to cleaning water pipe 33 and is provided in four units on the slump cone 24.

In some embodiments of the present disclosure, the slump plate operation mechanism 29 subsequently restores the dried slump plate 25 to its original position to commence the next batch of concrete slump and slump flow measurement.

### Method for Measuring Concrete Slump and Slump Flow

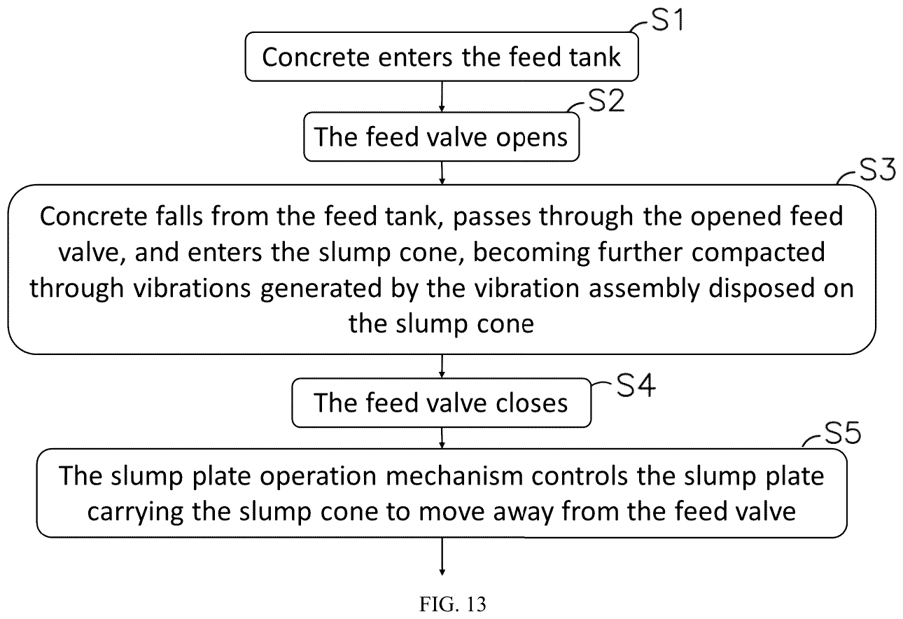

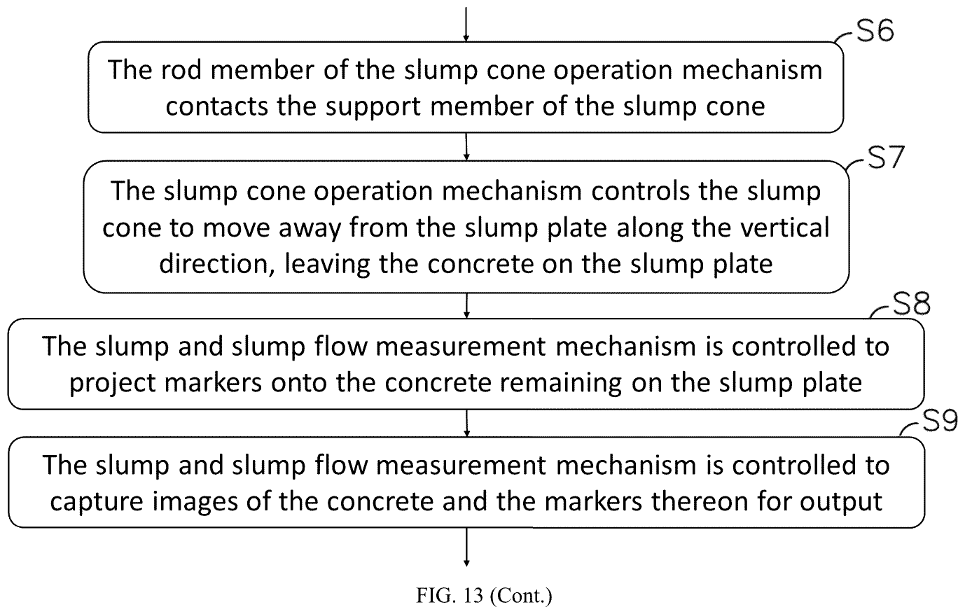

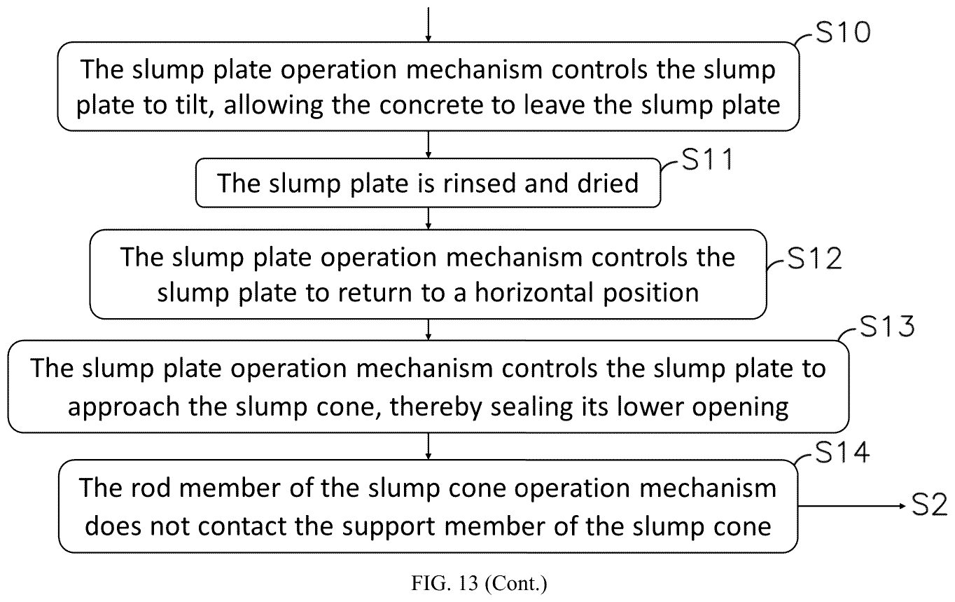

Next, referring to FIG. 13, it illustrates a flow diagram of the method for measuring concrete slump and slump flow as disclosed in the present disclosure.

For example, as shown in FIG. 1A or FIG. 8, in step S1, the concrete first enters the feed tank 21, while the feed valve 23 remains in a closed state.

Next, as shown in FIG. 5 or FIG. 9, in step S2 and step S3, the feed valve 23 will open (step S2), allowing the concrete to fall from the feed tank 21 through the opened feed valve 23 into the slump cone 24 (step S3). In some embodiments, the vibrator 22 of the present disclosure induces vibrations in the feed tank 21, ensuring the concrete to be evenly and reliably filled into the slump cone 24. In some embodiments, the slump cone 24 of the present disclosure is disposed with a vibration assembly 35, which compacts the concrete within the slump cone 24 through the vibrations generated by the vibration assembly 35. In some embodiments of the present disclosure, the feed tank 21 and the slump cone 24 have substantially identical volumes. In this case, the sampling amount of concrete substantially equals to the amount required for measurement.

Next, as shown in FIG. 2 or FIG. 10, in steps S4 and S5, after the slump cone 24 has been filled, the feed valve 23 will close (step S4). The slump plate operation mechanism 29 will then control the slump plate 25 to move downward, allowing the slump cone 24 to gradually move away from the feed valve 23.

Next, as shown in FIG. 3 or FIG. 11, in steps S6 and S7, the rod member 2721 of the slump cone operation mechanism 27 will be inserted into the support member 241 of the slump cone 24 (as shown in FIG. 3). The slump cone operation mechanism 27 will then control the slump cone 24 to move away from the slump plate 25 in a vertical direction, allowing the concrete to leave the slump cone 24 and remain on the slump plate 25.

Next, as shown in FIG. 6, in steps S8 and S9, the slump and slump flow measurement mechanism 28 projects markers onto the concrete 10 remaining on the slump plate 25 via the slump marker projector 283. The slump and slump flow measurement mechanism 28 then captures images of the concrete 10 and the markers thereon via the slump image capturer 284. In some embodiments of the present disclosure, the slump image capturer 284 of the slump and slump flow measurement mechanism 28 begins capturing images as the slump cone 24 moves away from the slump plate 25. The captured images are transmitted to a processor, which calculates the slump and/or slump flow of the concrete based on the captured images. Once the slump and/or slump flow of the concrete no longer change, image capturing is stopped.

Next, as shown in FIG. 4 or 12, in steps S10 and S11, the slump plate operation mechanism 29 controls the slump plate 25 to tilt, allowing the concrete to leave the slump plate 25 (step S10). Subsequently, the cleaning water pipe 31 and the drying air pipe 32 are employed to rinse and dry the slump plate 25, respectively (step S11).

As shown in FIG. 1A or FIG. 8, in steps S12, S13, and S14, the slump plate operation mechanism 29 controls the slump plate 25 to return to a horizontal position (step S12). Subsequently, the slump plate operation mechanism 29 controls the slump plate 25 to contact the lower portion of the slump cone 24, thereby sealing the lower opening of the slump cone 24 (step S13). At this time, the rod member 2721 of the slump cone operation mechanism 27 also returns to its original position and no longer contacts the support member 241 of the slump cone 24.

In at least one embodiment of the present disclosure, after the feed valve 23 is closed in step S4, the next batch of concrete can begin preparation for entry into the feed tank 21. Thus, upon completing steps S5 through S14, step S2 can be initiated directly to open the feed valve 23, thereby achieving the effect of rapid measurement of concrete slump and slump flow.

In at least one embodiment of the present disclosure, the method for measuring concrete slump and slump flow completes a full cycle in approximately 65 seconds. In other words, from loading concrete from the storage hopper 12 into the pre-mix concrete truck and simultaneously discharging concrete from the feed tank 21 into the slump cone 24, to subsequently performing slump and slump flow measurement by the slump and slump flow measurement mechanism 28, cleaning, drying, and waiting for the next discharge into the next pre-mix concrete truck while simultaneously discharging into the slump cone 24 for automatic sampling, all steps proceed without requiring intervention from mixing personnel. The entire process takes only approximately 65 seconds.

Given the foregoing, the apparatus and method for measuring concrete slump and slump flow as disclosed in the present application address numerous issues associated with manual operation of concrete, such as material retrieval, feeding, slump lifting, slump and slump flow measurement, cleaning, and the like. Through the design provided by the present disclosure, it is possible to achieve the effectiveness of automatic material retrieval, automatic feeding into the slump cone, automatic slump lifting, automatic slump and slump flow measurement through image capturing, automatic output of captured images and information, automatic slump and slump flow detection, and automatic cleaning of the slump plate. These features enable mixing personnel at the mixing station to inspect the received information in real time. Furthermore, with the slump cone operation mechanism, slump plate operation mechanism, and slump and slump flow measurement mechanism as disclosed herein, the effectiveness of making the slump and slump flow measurement process faster and more accurate is achieved, thereby obtaining real-time and precise slump and slump flow data and ensuring the quality and safety of structures.

For a person having ordinary skill in the art, it is evident that the fundamental principles described herein may be implemented in various ways as technology advances. Therefore, the specific embodiments are not limited to the examples described above; rather, variations may occur within the scope of the appended claims.

The specific embodiments described above can be combined with each other in any manner. Multiple specific embodiments may be combined to form additional specific embodiments. The methods disclosed herein may include at least one specific embodiment described above. It should be understood that the aforementioned benefits and advantages may pertain to a single specific embodiment or to multiple specific embodiments. The specific embodiments are not limited to addressing any or all of the stated issues, nor are they limited to achieving any or all of the stated benefits and advantages.

What is claimed is:

1. An apparatus for measuring concrete slump and slump flow, comprising:
a frame;
a feed valve disposed within the frame;
a feed tank disposed above the feed valve and configured to hold concrete to be measured;
a slump cone disposed below the feed valve, the slump cone having an upper opening aligning with the feed valve, allowing the concrete to fall from the feed tank and pass through an opened feed valve into the slump cone;
a slump plate disposed below the slump cone, the slump plate configured to seal a lower opening of the slump cone, thereby retaining the concrete within the slump cone;
a slump cone operation mechanism disposed on the frame and connected to the slump cone, the slump cone operation mechanism configured to move the slump cone away from the slump plate along a direction perpendicular to the plane of the slump plate, allowing the concrete to leave the slump cone and remain on the slump plate;
a slump and slump flow measurement mechanism configured to project a marker onto the concrete remaining on the slump plate and to capture images of the concrete and the marker; and
a slump plate operation mechanism connected to the slump plate and configured to tilt the slump plate, allowing the concrete to leave the slump plate.

2. The apparatus according to claim 1, wherein the slump and slump flow measurement mechanism comprises a slump marker projector configured to project a slump marker, a slump flow marker projector configured to project slump flow markers, a slump image capturer, and a slump flow image capturer.

3. The apparatus according to claim 2, wherein the slump flow markers are two straight lines intersecting each other perpendicularly and intersecting side edges of the concrete remaining on the slump plate, respectively, and the slump marker is a straight line intersecting an upper-end edge of the concrete remaining on the slump plate.

4. The apparatus according to claim 1, further comprising a processor configured to receive images captured by the slump and slump flow measurement mechanism and to calculate the slump and/or the slump flow of the concrete, respectively, based on the images with the concrete and the slump marker and the slump flow markers thereon.

5. The apparatus according to claim 1, wherein the slump cone operation mechanism comprises a vertical actuator connected to the frame and having an actuating rod, and a horizontal actuator connected to the vertical actuator, wherein the horizontal actuator includes a rod member, the slump cone is disposed with a support member, the rod member of the horizontal actuator is configured to contact the support member of the slump cone to support the slump cone, and the vertical actuator adjusts a length of the actuating rod to move the slump cone toward or away from the feed valve.

6. The apparatus according to claim 1, wherein the slump plate operation mechanism comprises a central actuator connected to a position near the center of the slump plate and an edge actuator connected to an edge of the slump plate, wherein the slump plate operation mechanism moves the slump cone carried on the slump plate toward or away from the feed valve along a vertical direction by the central actuator and the edge actuator.

7. The apparatus according to claim 1, further comprising a cleaning water pipe and a drying air pipe disposed on at least one side of the slump plate, and a discharge chute disposed adjacent to a corresponding side of the slump plate.

8. The apparatus according to claim 1, further comprising a mixing bucket disposed above the feed tank and a load cell disposed on the mixing bucket, wherein the volume of the feed tank is substantially equal to the volume of the slump cone.

9. The apparatus according to claim 1, further comprising a vibration assembly disposed on the slump cone, allowing the concrete to enter the slump cone from the feed tank and to be compacted by vibrations of the vibration assembly.

10. A method for measuring concrete slump and slump flow by using the apparatus according to claim 1, comprising:
introducing the concrete into the feed tank;
controlling the feed valve to open, allowing the concrete to fall from the feed tank, pass through the opened feed valve, and enter the slump cone, wherein the slump plate seals the lower opening of the slump cone to retain the concrete within the slump cone;
controlling, by the slump plate operating mechanism, the slump plate, allowing the slump cone carried on the slump plate to move away from the feed valve;
controlling, by the slump cone operation mechanism, the slump cone, allowing the slump cone to move away from the slump plate along a direction perpendicular to the plane of the slump plate, and allowing the concrete to leave the slump cone and remain on the slump plate;
controlling the slump and slump flow measurement mechanism to project a marker onto the concrete remaining on the slump plate;
controlling the slump and slump flow measurement mechanism to capture images of the concrete and the marker; and
controlling, by the slump plate operation mechanism, the slump plate to tilt, allowing the concrete to leave the slump plate.

11. The method according to claim 10, wherein controlling the slump and slump flow measurement mechanism to project markers onto the concrete remaining on the slump plate further comprises employing the slump and slump flow measurement mechanism to project a slump marker and slump flow markers onto the concrete on the slump plate, wherein the slump flow markers are two straight lines intersecting each other perpendicularly and intersecting side edges of the concrete on the slump plate, respectively, and the slump marker is a straight line intersecting an upper-end edge of the concrete on the slump plate.

12. The method according to claim 10, further comprising employing the slump and slump flow measurement mechanism to start capturing images when the slump cone moves away from the slump plate, and transmitting the captured images to the processor, allowing the processor to calculate the slump and/or slump flow of the concrete based on the images.

13. The method according to claim 10, wherein the slump cone operation mechanism controlling the slump cone to move away from the slump plate along the direction perpendicular to the plane of the slump plate further comprises:
subjecting the rod member of the horizontal actuator of the slump cone operation mechanism to contact the support member on the slump cone; and
adjusting a length of the actuating rod of the vertical actuator of the slump cone operation mechanism, allowing the slump cone to move away from the slump plate.

14. The method according to claim 10, further comprising, after completing image capturing, controlling the cleaning water pipe to discharge water to rinse the slump plate, allowing the water and the concrete to flow into the discharge chute, and controlling the drying air pipe to discharge air to dry the slump plate.

15. The method according to claim 14, further comprising:
after the concrete leaves the slump plate and the slump plate is rinsed and dried, controlling, by the slump plate operation mechanism, the slump plate to return from a tilted position to a horizontal position;
controlling, by the slump plate operation mechanism, the slump plate to move toward the slump cone to seal the lower opening of the slump cone; and
subjecting the rod member of the horizontal actuator of the slump cone operation mechanism to return to a position without contacting the support member on the slump cone.

16. The method according to claim 10, further comprising:
mixing the concrete in the mixing bucket; and
controlling the mixing bucket to tilt, allowing the concrete to fall into the feed tank, wherein the volume of the feed tank is substantially equal to the volume of the slump cone.

17. The method according to claim 10, wherein the time from the concrete falling from the feed tank to a next batch of the concrete falling from the feed tank is approximately 65 seconds, and the method is performed in an automatic and continuous manner.

18. The method according to claim 10, further comprising compacting the concrete entering the slump cone from the feed tank by vibrations of a vibration assembly disposed on the slump cone.
